(19) United States
(12) Patent Application Publication
(10) Pub. No.: US 2026/0286066 A1
(43) Pub. Date: Sep. 24, 2026

(54) CONVERSION OF LIGNIN INTO BIO-PHENOLS, LIGNIN-BASED EPOXIDES, AND BIO-BASED EPOXY RESIN

(71) Applicant: SPERO RENEWABLES, LLC, Goleta, CA (US)

(72) Inventors: Baoyuan LIU, Goleta, CA (US); Ian M. KLEIN, Goleta, CA (US); Shou ZHAO, Goleta, CA (US)

(21) Appl. No.: 19/472,545

(22) PCT Filed: Apr. 4, 2024
(86) PCT No.: PCT/US2024/022955

§ 371 Date: Oct. 3, 2025

Related U.S. Application Data
(60) Provisional application No. 63/494,317, filed on Apr. 5, 2023.

(51) Int. Cl. C08H 7/00

(52) U.S. Cl. CPC C08H 6/00

(57) ABSTRACT

A method for depolymerizing lignin includes contacting lignin with a catalyst, and based on the contacting, catalytically depolymerizing at least a portion of the lignin into low molecular weight fragments. The low molecular weight fragments have Mₙ of less than or equal to 350 Da, and the low molecular weight fragments comprise unbound phenolic hydroxyl groups.

### CROSS-REFERENCE TO RELATED APPLICATIONS

This application is a filing under 35 U.S.C. 371 as the National Stage of International Application No. PCT/US2024/022995, filed on Apr. 4, 2024, entitled, “CONVERSION OF LIGNIN INTO BIO-PHENOLS, LIGNIN-BASED EPOXIDES, AND BIO-BASED EPOXY RESIN,” which claims the benefit of and claims priority to U.S. Provisional Patent Application No. 63/494,317 filed on Apr. 5, 2023, and entitled “CONVERSION OF LIGNIN INTO BIO-PHENOLS, LIGNIN-BASED EPOXIDES, AND BIO-BASED EPOXY RESIN,” both of which are incorporated herein in their entirety by reference for all purposes

### Cross Reference to Related Applications

This application claims priority to U.S. Provisional Patent Application No. 63/494,317 filed on Apr. 5, 2023, and entitled “CONVERSION OF LIGNIN INTO BIO-PHENOLS, LIGNIN-BASED EPOXIDES, AND BIO-BASED EPOXY RESIN,” which is incorporated herein in its entirety by reference.

### FIELD OF THE DISCLOSURE

This disclosure relates to a method for converting technical lignin generated from a range of pulping and lignin removal methods into bio-based phenolic molecules (bio-phenols, or bio-based multifunctional phenols) that are suitable for making bio-based epoxides and are further upgradable to bio-based epoxy resins. More specifically, this disclosure describes a stepwise procedure which can include catalytic lignin depolymerization, sulfur removal, catalyst regeneration, epoxidation of bio-phenols, and resin synthesis. By which, the bio-phenols are produced from lignin through the catalytic depolymerization under a reductive condition using solvents. Sulfur content can also reduce during the reaction. Then bio-based epoxides are made from a reaction between bio-phenols and epichlorohydrin. Finally, the bio-based (or lignin-based) epoxy resin can be synthesized by mixing bio-based epoxides and hardeners (curing agent). In particular, the synthesis of bio-based epoxy resin integrates both elastomer and rigid polymer composite with a variety of formulations.

### BACKGROUND

Lignocellulose is sustainably produced by photosynthesis which converts the solar energy to stored chemical energy on Earth. It is the most abundant renewable organic feedstock for chemical production. An annual production of 1.4-1.6 billion tons of lignocellulosic biomass can be achieved by 2030. Lignocellulosic biomass is composed of three major biological materials: cellulose (50%), hemicellulose (25%), and lignin (25%). They are the major building blocks in the architecture of plant cell walls. Cellulose and hemicellulose are polysaccharides while the lignin is an aromatic polymer. Notably, lignin is the largest renewable source of natural aromatic chemicals. Nowadays, the use of lignocellulosic polysaccharides has been successfully commercialized to produce bioethanol. In contrast, the utilization of lignin is still under development. For instance, paper and pulp industries produce 50-70 million tons of lignin every year, however 98% of it is still consumed by direct combustion. Burning lignin only utilizes the heat energy of hydrocarbon while the chemical values of aromatics are fully discarded.

The catalytic conversion of lignin over transition metals has been studied in both research articles and patents (doi.org/10.1039/D2GC01278B, doi.org/10.1039/C5OB02212F, and US2020/0317593A1). Transition metals, such as Ni, Co, Pd, Ru, Pt, and Ir, have been investigated to be effective metallic catalyst for lignin depolymerization. However, previous work in catalytic conversion of lignin has focused on conversion of lignin to produce low molecular weight monomeric products or conversion of lignin to products with a wide range of molecular weights, neither of which are well suited for direct epoxidation nor epoxy resin synthesis.

Epoxy resins are essential to our modern life which contribute to a wide range of applications, such as electronics, automobiles, manufacturing, adhesives, paints/coatings, aerospace, and marines. The global epoxy resin market was valued at U.S. dollars (USD) $ 13 billion in 2021 and is projected to grow at a compound annual growth rate (CAGR) of 7.21% to reach USD 23.4 billion by 2030. Of which, 85% of all epoxy resins are made from reaction between bisphenol-A (BPA) and epichlorohydrin (ECH). BPA is an organic compound that contains two phenol functional groups. It is manufactured by an acid catalyzed condensation of phenol and acetone which are stemming from oil refining of petrochemicals. To date, due to the toxicity and health effects of BPA to human body, alternatives of BPA and BPA-free epoxy resins are catching more attention on both scientific and economic prospects. On one hand, the BPA-free coating market has grown to be USD $7 billion in 2022 and is estimated to reach USD $ 13 billion by 2032, increased at a CAGR of 6.5%. On the other hand, many studies have proven lignin is a great source to produce alternative chemicals of BPA for epoxy resin synthesis. Gioia, et al. (doi.org/10.1021/acs.biomac.0c00057) demonstrated the main monomeric units of lignin, phenolic monomers, have ability to substitute BPA to produce epoxy resin with good thermal and mechanical performance. Similarly, Zhao, et al. (doi.org/10.1021/acs.biomac.5b00670 and doi.org/10.1021/acs.macromol.8b01976) presented lignin-based molecules having potential for producing epoxy resin with great mechanical properties and novel recyclability. Beyond that, Zhen, et al. (doi.org/10.1016/j.ijbiomac.2021.03.203) suggested the lignin-based epoxy resin could be a better flame retardant than a BPA-based epoxy resin. Additionally, compared to petro-based chemicals, using lignin as feedstock for resin production is sustainable, environmentally friendly, and cost-efficient. Therefore, utilization of kraft lignin for epoxy resin is of great interest.

However, the global lignin-based (or bio-based) epoxy market is still small which was investigated to be USD $ 50 million in 2019. It is expected to grow at a CAGR of 12% between 2020-2027 to reach USD $120 million. In addition, information on how to carry out the direct conversion of kraft lignin into epoxy resin is still limited. For instance, the publication (U.S. Pat. No. 9,006,369B2) disclosed a method to produce water-based lignin epoxy resin. Another example of publication (US20160102170A1) disclosed a method to make epoxy resin from modified lignin in mixture with acid anhydrides and diols or polyols. Similarly, the publications (US2012/0148740A1, US2011/0024168A1, U.S. Pat. No. 9,856,346B2) also require acid anhydride and polyol as the parts for lignin-based epoxy resin. The publications (U.S. Pat. No. 4,111,928A and 4,265,809A) describe the kraft lignin, however, required multi-step alterations for making lignin-based epoxy resin. Besides the lack of published information, the accessible lignin products on today's market for bio-based epoxy resins are also limited. For instance, one of the key players, Gougeon Brothers, who is leading several brands such as West System, Pro-Set, and Entropy Resins, etc. Their bio-based products are mainly focusing on the application of hardeners for resin synthesis. Moreover, the bio-based products are mainly sourced from vegetable oils, soybean extracts, cashew nutshell extracts, and bio-glycerol.

### SUMMARY

In some embodiments, a method for depolymerizing lignin comprises contacting lignin with a catalyst, and based on the contacting, catalytically depolymerizing at least a portion of the lignin into low molecular weight fragments. The low molecular weight fragments have Mₙ≤350 Da, and the low molecular weight fragments comprise unbound phenolic hydroxyl groups.

In some embodiments, a method comprises contacting technical lignin with one or more organic solvents, fractioning the technical lignin based on the contacting to form lignin fractions, and epoxidizing one or more of the lignin fractions for resin synthesis to form lignin epoxide.

In some embodiments, a method comprises providing lignin-derived epoxides, curing the lignin-derived epoxides with one or more amine hardeners, and producing bio-based elastomers from the lignin-derived epoxides cured with the amine hardeners. The one or more amine hardeners comprise Jeffamine-230, IPDA, DETA, and DDPS, and the curing occurs at a temperature between about 25 C to about 120 C.

In some embodiments, a reactor system comprises a lignin dissolution vessel configured to contact lignin with a solvent, a catalytic reactor in fluid communication with the lignin dissolution vessel, and a separator configured to separate product bio-phenols from the solvent are recycle the solvent to the lignin dissolution vessel. The catalyst reactor comprises a catalyst bed configured to contact soluble lignin from the lignin dissolution vessel at elevated temperature and pressure.

These and other features will be more clearly understood from the following detailed description taken in conjunction with the accompanying drawings and claims.

### BRIEF DESCRIPTION OF THE FIGURES

For a more complete understanding of the present disclosure, reference is now made to the following brief description, taken in connection with the accompanying drawings and detailed description:

FIG. 1 illustrates a GPC analysis of (a) bio-phenols obtained from 90% MeOH medium give the most uniformed and the least Mₙ which is around 300 Da; (b) bio-phenols obtained from 90% MeOH with 10% acetone co-solvent medium contain both high Mₙ (minor) and low Mₙ (major) fractions which results the highest yield of bio-phenols up to 90%; and (c) the heavy bio-phenols washed from solid residue contain mainly the very high Mₙ fraction between 35000-41000 Da.

FIG. 2 illustrates a model reaction system which equipped with a jacked stainless-steel filter reactor coupled with a packed-bed reactor for catalysis. An evaporation system is attached to the reactor to separate the bio-phenols from product stream and condense/recycle the solvents.

FIGS. 3A and 3B illustrate 2D ¹H/¹³C HSQC NMR spectra. (a) 2D HSQC NMR spectrum of feedstock kraft pine lignin before the catalytic reaction which contains lignin linkages such as β-O-4, β-β, and β-5. (b) 2D HSQC NMR spectrum of bio-phenols derived from kraft pine lignin which indicates most of the lignin linkages are effectively cleaved during the catalytic depolymerization reaction described in this invention.

FIGS. 4A and 4B illustrates the results of DMA analysis of epoxy resins made from 100% bio-based epoxides and from blending 75% bio-based epoxides with 25% DGEBA. Both bio-based epoxy resins are cured by DETA. (a) T_{g} of the epoxy resins determined by DMA analysis. By which, the resin made from 100% lignin-derived epoxides (red colored) give T_{g} at around 60° C. while the 75/25 blended epoxy resin (black colored) give T_{g} at around 90° C. (b) The storage modulus of both epoxy resins is measured to be ≥1 gigapascal (GPa).

### DETAILED DESCRIPTION

The present disclosure relates to a complete process starting from technical lignin, including catalytic conversion of lignin into bio-based phenolic molecules (bio-phenols), catalyst recycling/regeneration, epoxidation of the bio-phenols, and formulated epoxy resin synthesis. Technical lignin refers to lignin which has been removed or extracted from biomass using a variety of techniques including but not limited to steam explosion, acid or base treatment, organosolv treatment, kraft pulping, or extraction with ionic liquids, or other known methods for lignin removal from biomass. In some cases, technical lignin produced by kraft pulping, or other techniques can be further purified by methods such as the LignoBoost process. The LignoBoost process includes a precipitation at a high pH followed by filtration and purification at low pH to improve the purity of the lignin, where the process can result in the addition of sulfur in some aspects. One source of technical lignin for upgrading in the present disclosure is kraft lignin made from pine wood pulping which has been further processed using the LignoBoost process. This source of lignin is easily accessible from paper companies in large quantities. Due to the nature of LignoBoost technology, the processed kraft pine lignin contains several percentages of sulfur. The presence of sulfur within kraft lignin is mainly functionalized as organic sulfur, including but not limited to disulfide, sulfate, hydrogen sulfide, and thiols that attaching to lignin carbons.

The catalytic conversion of lignin can be related to a reductive depolymerization reaction catalyzed by transition metals supported on activated carbon or metal oxides. In the present disclosure, transition metals including, but not limited to, Ni, Co, Pd, Ru, Pt, and Ir can be deposited on supports including but not limited to carbon (C), alumina (Al₂O₃), and a variety of other metal oxides, and to be used as an effective catalyst for lignin depolymerization. Nickel (Ni), an earth abundant and low-cost metal can be used for the catalyst to depolymerize kraft pine lignin. Both activated carbon (C) and alumina (Al₂O₃) supported Ni catalysts, Ni/C and Ni/Al₂O₃, have been found to be effective catalysts for lignin depolymerization in the present disclosure. The Ni catalysts described in this disclosure can be purchased from commercial catalyst suppliers or synthesized using known procedures and contain at least 10% Ni content per gram of the catalyst, although lower or higher Ni loading can be used in the catalyst as well. Both Ni/C and Ni/Al₂O₃ were found to effectively depolymerize technical lignin, additionally both catalysts were recyclable through multiple cycles of lignin depolymerization. However, Ni/Al₂O₃ shows better performance after regeneration under a simple thermal process. Unlike using single lignin monomeric phenols or a mixture of multi lignin derived monomers, the present disclosure confirmed the ability to epoxidize the lignin fragments (bio-phenols) directly generated from Ni catalyzed depolymerization. This is because the lignin fragments made in this disclosure contain active phenolic functional groups and thus is active for epoxidation reaction to produce bio-based epoxides. This finding simplifies the overall process of converting kraft lignin into epoxy resin. By which, the present disclosure is able to overcome the difficulty of narrowing the selectivity down to produce specific lignin monomers and skip the complicate extraction of single lignin monomer out of a complex reaction mixture. Therefore, the additional advantages of the present disclosure are the reduced cost and energy/chemical input for product separation and herein resulting the reduced release of green-house gas. Followed by the epoxidation of bio-phenols, the epoxy resins are made. In the present disclosure the lignin-based (or bio-based) epoxy resin in this work can be formulated for a variety of applications, including but not limited for making elastomers, coatings, and rigid polymer composites, etc.

In some embodiments, technical lignin including but not limited to kraft lignin or LignoBoost lignin can be directly epoxidized without any pre-treatment or pre-reaction. By which, the technical lignin including but not limited to kraft lignin or LignoBoost lignin can be mixed with ECH in a reaction vessel and the mixture is heated to desired reaction temperature and held at the temperature till the completion of epoxidation. The produced lignin-based (or bio-based) epoxides is a high viscous liquid at the reaction temperature, and while at room temperature, may not be free-flowing. Nevertheless, the epoxides made in such way are useful for epoxy resin synthesis in many formulations including but not limited to formulations where it is acceptable to pre-heat the epoxides mixture to 30-50° C. to lower the viscosity prior to mixing with curing agents, which can include, but are not limited to, Jeffamine-230 (Jeffamine D-230 polyetheramine), isophorone diamine (IPDA), diethylenetriamine (DETA), and diaminodiphenyl sulfone (DDPS), etc.

In some embodiments, the technical lignin can be separated into fractions and have at least a portion of any sulfur removed using a sequential wash or dissolution process or step. In some embodiments, the technical lignin including but not limited to kraft lignin or LignoBoost lignin is first fractionated by sequential washes with different solvents, including but not limited to water, ethanol (EtOH), acetone, and/or ethyl acetate (EtOAc), etc. The sequential washing treatment can be used to collect the organic soluble fractions of the lignin into different solvents while leaving the insoluble portion separated as solid residue by filtration. By which the sulfur content of the technical lignin can be reduced by 10-90% in the soluble lignin fractions. Both the soluble lignin fractions and solid residue are useful for making epoxides and epoxy resins. Notably, the distributions of molecular weight (Mₙ, which is the number average molecular weight) of dissolved lignin fractions are different in each solvent. For instance, dissolved lignin fraction in EtOAc shows average Mₙ at around 200-1000 Dalton (Da) while the dissolved lignin fraction in EtOH and acetone give higher Mₙ, 1000-7000 and >10000 Da, respectively. The insoluble fraction of kraft lignin shows the highest average Mₙ which can be up to 65000 Da. At all fractions, the lignin is stills considered as intact kraft which retained its original polymeric framework and thus can be converted into epoxides and epoxy resin the same way as using kraft lignin directly.

The kraft lignin or separated fractions can also be depolymerized to some degree to produce bio-phenols. In some embodiments, the technical lignin, including but not limited to, kraft lignin or LignoBoost lignin can be treated with a catalyst such as Ni/C, Ni/Al₂O₃, recycled Ni/C, recycled Ni/Al₂O₃, and/or regenerated Al₂O₃ under depolymerization conditions to produce bio-phenols. The produced bio-phenols are lignin fragments instead of any specific known phenolic molecules. The depolymerization reaction can use solvents including alcohols such as ethanol or methanol or other organic solvents such as acetone.

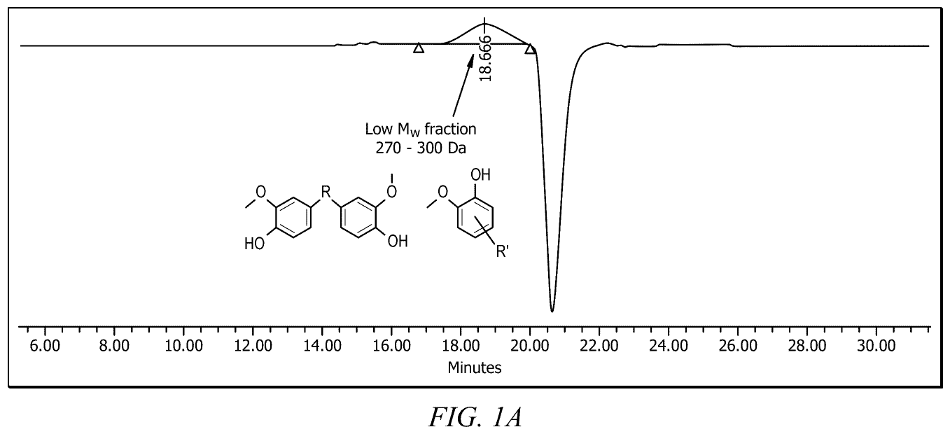

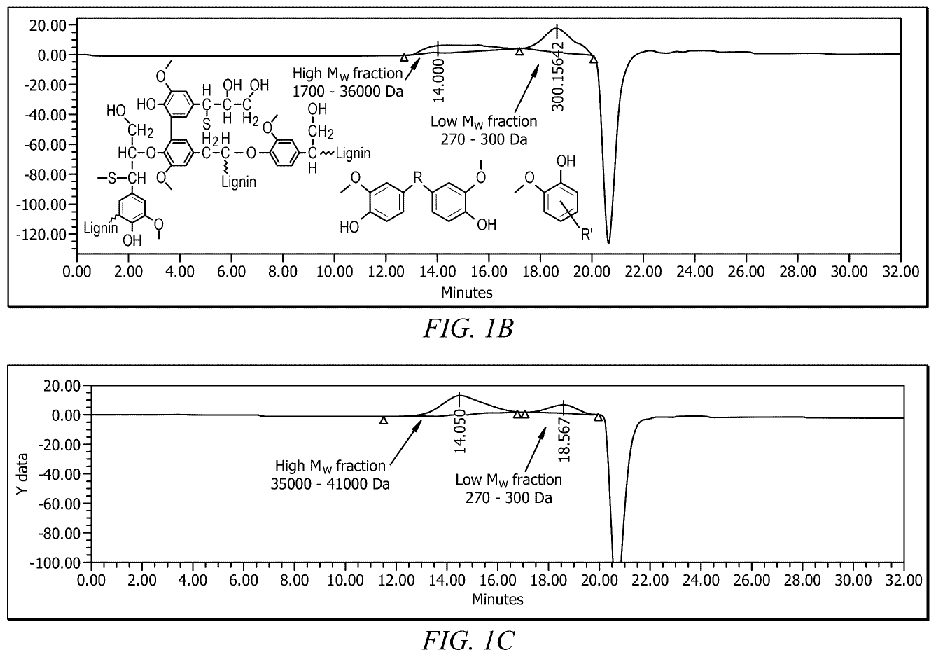

The organic solvent may be present in a volume percentage between about 10% to about 95% of each component, and in some aspects, water or another solvent may be present. For example, the depolymerization reaction can use a solvent including, but not limited to, 90% EtOH (EtOH/H₂O 9:1 volume ratio—v/v), 90% methanol (MeOH, MeOH/H₂O 9:1 v/v), 90% EtOH with 10% acetone (v/v) and 90% MeOH with 10% acetone (v/v). The produced bio-phenols from different solvents or co-solvent mixtures are all useful for making corresponding epoxides and epoxy resins. This is due to the abundance of hydroxy groups/numbers (OH_{number}) within the bio-phenols which could be ranging from 2 -12 mmol/g_{bio-phenol} (where mmol is millimolar) or about 6-8 mmol/g_{bio-phenol}. However, the overall yield and the average Mₙ of bio-phenols can vary for different reaction medium. For example, technical lignin reacted with a catalyst and 90% MeOH reaction medium yields a highly uniform product mixture and a low average Mₙ of bio-phenols at around 300 Da (FIG. 1). In contrast, when technical lignin is reacted with a catalyst under the same reaction conditions using a reaction solvent of 90% EtOH with 10% acetone medium yields a broader and higher Mₙ of bio-phenols which is ranging from 270 and is up to 40000 Da. A high yield of bio-phenols from technical lignin reacted with a catalyst can be obtained from a reaction solvent of 90% MeOH with 10% acetone which 90wt % of input technical lignin is converted into bio-phenols. A yield of bio-phenols can be around 60wt % in a 90% EtOH medium. Notably, the epoxides made from bio-phenols show lower viscosity (1-1500 cSt at 20° C., cSt: centistokes) than the epoxides made from intact kraft, or technical lignin and organic solvent extracted lignin fractions (≥1500 cSt at 20° C.). The epoxides made from the described bio-phenol mixtures are free-flowing at room temperature (20° C.) and can be easily mixed with curing agents. Furthermore, the epoxides made from bio-phenols obtained in 90% MeOH solvent have the lowest viscosity at room temperature compared to other epoxides described in this disclosure. Therefore, while not wishing to be limited by theory, it can be hypothesized that the viscosity of epoxides is related to the Mₙ of its corresponding bio-phenols which the lower average Mₙ yielding less viscous epoxidized products.

In some embodiments, the catalyzed lignin depolymerization reactions are carried under different pressures of inert gasses such as nitrogen or argon, at different temperatures, and over different reaction times. With many cases, the optimal gas pressure has been determined to be between 200-500 pounds per square inch (psi) at room temperature (20° C.) in a batch style reaction system. In many cases, the temperature for lignin depolymerization reaction can be between 100-250° C., or 160-240° C. In many cases, the lignin depolymerization reaction time in a batch reactor can be between 0.1-25 hours, or 6-12 hours (h). The reaction conditions can balance the yield of bio-phenols, cost of energy, and use of solvents and catalysts. Notably, the loading of catalysts has been also tested for this disclosure. By which, based on the input mass of technical lignin, a minimum of 10% catalyst is preferred in batch style reactions. The produced bio-phenols obtained from all the described reaction conditions have been tested to be useful for making epoxides and epoxy resins.

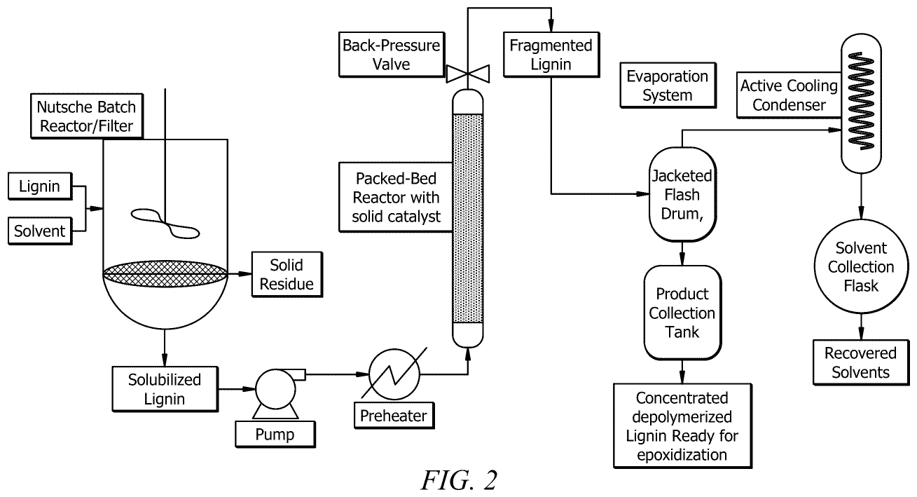

In some embodiments, the catalytic depolymerization reaction of kraft lignin can be piloted in the reaction system in FIG. 2. By which, the technical lignin is first loaded with the mentioned solvents in this disclosure into a jacked stainless steel filter reactor (J-reactor). The mixture in the J-reactor is stirred my magnetic stirring between 100-2000 revolutions per minute (rpm) to ensure a good dissolving of lignin in the solvent. The solubilized lignin is filtered through the filter equipped on the bottom of the J-reactor while the insoluble lignin residues are removed from the reaction system. The solubilized mixture is then pressurized to 50-2000 psi and pre-heated to desired reaction temperature between 95-300° C. and pumped over a packed catalyst bed. The catalytic lignin depolymerization reaction is carried out in the packed-bed reactor (P-reactor) when the hot reaction medium reaches the catalyst in P-reactor. The catalyst packed in the P-reactor can be easily regenerated in situ with proper heating under air flow. The products stream can then be depressurized after reaction in P-reactor and purged into an evaporation setup where the bio-phenols are collected by evaporating the solvents. The concentrated bio-phenols are ready for epoxidation reaction while the hot solvent steam can be recovered by passing through an active cooling condenser and then can be recycled back to the J-reactor.

In some embodiments, unaltered technical lignin can be used as feedstock for epoxidation reaction to react with ECH. In some embodiments, the technical lignin fractionated by selective solubilization in different organic solvents is used as feedstock for epoxidation reactions. In some embodiments, the bio-phenols obtained from different catalytic reactions of technical lignin including but not limited to kraft lignin or LignoBoost lignin with catalysts including but not limited to Ni/C or Ni/Al₂O₃ are used as feedstock for epoxidation. In some embodiments, the ECH was bio-based which is manufactured from glycerin commercially and thus the resulting epoxides is 100% bio-based (100% bio-content). Nevertheless, the condition of epoxidation reaction with ECH remains the same. By which, the feedstocks are physically mixed with ECH in reaction vessel equipped with continuous stirring. A small amount of tetrabutylammonium bromide (TBAB) is used as catalyst for epoxidation reaction in this disclosure. The desired reaction temperature of epoxidation is set at 30-120° C. or about 80° C. The ratio of ECH to feedstock materials is set to 10:1 by weight ratio while the loading ration of TBAB is 10% by weight of feedstock. Once the reaction mixture reaches the desired temperature, the reaction is then held for 0.5-6 h to achieve the completion of epoxidation. After that, 5% of sodium hydroxide (NaOH, based on weight of ECH loading) is added with small amount of water to the reaction mixture. At this point, the reaction is held at 30-120° C. or about 80° C. for another 1-5 h. Finally, the reaction mixture is cooled to room temperature with addition of a solvent such as acetone. The volume of solvent such as acetone added to the reaction can be between 1-25 or 10-15 times of the overall reaction volume of ECH. The addition of solvent such as acetone is to quench the reaction and precipitate the NaCl out of the organic products stream. Following by a filtration and evaporation of excess ECH and solvent such as acetone, the neat lignin-based (or bio-based) epoxides are obtained. As described above, depending on the feedstock materials, the bio-based epoxides can have a variety of viscosity at 20° C. ranging from a free-flowing liquid to a very sticky gel-like material that does not flow at room temperature. Aside from the viscosity, the appearance of the bio-based epoxides is almost the same which is between black and dark brown colored.

In some embodiments, the epoxy resin is synthesized with 100% of bio-based epoxides while in some embodiments the resin is made of a blend with lignin-based epoxides and commercial BPA-based epoxides (bisphenol A diglycidyl ether abbreviated as DGEBA herein), or any number of other commercially available epoxides. Bending of the describe bio-based epoxides with commercially available epoxides can be used to achieve different mechanical properties of the synthesized epoxy resin. Depending on the needs of epoxy resin, the ratio of blending lignin-based epoxides vs. DGEBA can be ranging from addition of 0% DGEBA-95% DGEBA or other commercially available epoxide. Similarly, several curing agents have been used in this disclosure which confirmed the feasibility of our lignin-based epoxides that can be cured with a wide range of curing agents. The curing agents have been described above, including but not limited to Jeffamine-230, IPDA, DETA, and DDPS. The blended lignin-based epoxides with DGEBA have also tested to be cured with the curing agents to produce epoxy resins. The resins made from this disclosure share a comparable appearance which is nearly black and dark brown colored. In addition, this disclosure produces low-odor epoxy resin from technical lignin. Beyond that, elastomeric epoxy resin can be made with the epoxides derived from bio-phenols obtained in 90% MeOH solvent or other solvents and finally cured with Jeffamine-230 or other curing agents suitable for elastomer production. In contrast, either blending with DGEBA or curing with IPDA and DETA could effectively reduce the elasticity and increase the hardness, glass transition temperature (T_{g}), and storage modulus. The measured T_{g} has a broad range which is from <20° C. and up to 140° C. The measured storage modulus also gives a wide range which is between 0.1-3 GPa.

Below, exemplary methods and embodiments with be described in detail. The contents of this disclosure may be embodied in various forms without being limited to the exemplary methods and embodiments. Description of steps well known to those skilled in the art are omitted for clarity.

The present process relates to the treatment using water and organic solvents to wash the kraft lignin and thus fractionate different lignin parts having identical solubility in each solvent. The wash can be sequential or one solvent at each time with fresh kraft lignin.
(1) Load technical lignin into a reaction vessel with 1-15 or 2-5 times volume of solvent per g of input technical lignin.
(2) Warm the mixture to 20-95° C. with continuous stirring between 200-500 rpm for 0.1-9 h.
(3) Filter the mixture thus the dissolved lignin fraction is collected in the filtrate.
(4) Dry the filtrate under vacuum using a rotavapor or evaporator. The drying condition can vary depending on the use of solvent.
(5) The dried lignin fraction is collected as solid after the complete removal of solvent.

In some embodiments, the collected lignin fractions are directly used for epoxidation reaction while in some embodiments the collected lignin fractions are analyzed by gel permission chromatography (GPC), 2-dimensional proton/carbon heteronuclear signal quantum correlation nuclear magnetic resonance spectroscopy (2D ¹H/¹³C HSQC NMR), and elemental analysis. The analytical procedures are well-known procedures which are not specified in this disclosure.

The present disclosure relates to a method to convert technical lignin including but not limited to kraft lignin or LignoBoost lignin into bio-phenols. Several catalysts, solvents, conditions have been investigated in this disclosure. A typical catalyzed lignin depolymerization reaction is carried out in a stainless-steel reactor that can tolerate high pressure and high temperature. For example, the reactor designed by Parr Instrument Company based in Moline, IL. For this disclosure, the reaction system includes but is not limited by batch reactor system, flow reactor system, Nutsche batch system, or a packed-bed reactor system, etc. (FIG. 2). When operated in a batch reactor the reaction steps can optionally include any of the following steps:
(1) Load the technical lignin into reactor vessel following by mixing with 1-30 wt % of catalyst such as Ni/C or Ni/Al₂O₃ and desired solvent, such as 0.1-90% EtOH (v/v with water), 0.1-90% EtOH with 0.1-25% acetone, 0.1-90% MeOH, and 0.1-90% MeOH with 0.1-25% acetone. The volume of solvent is between 1-20 times of lignin weight in unit of liter per kilogram (L/kg). The loading of catalyst is based on the mass of input lignin, i.e., 100 g catalyst is used for 1 kg kraft lignin.
(2) Stir the reaction mixture at 50-1000 rpm.
(3) Purge the reactor system with an inert gas to minimize oxygen and charge 200-500 psi inert gas such as N₂ prior to heating.
(4) Once the reaction system is well isolated and pressurized, bring the reaction temperature to be between 100-250° C. for a desired reaction time (0.1-24 h).
(5) After the completion of reaction, the mixture is cooled to room temperature.
(6) The cooled reaction mixture is filtered, and the bio-phenol products are collected in filtrate.
(7) Transfer the filtrate into a flask and remove the solvent from the bio-phenol products by distillation or evaporation.
(8) The dried bio-phenol products are collected after removal of solvent.
(9) Some solid residues are also collected from the filtration in step (6) of this method.
(10) The solid is dried in oven at around 50-70° C. with less than 2% of moisture content.

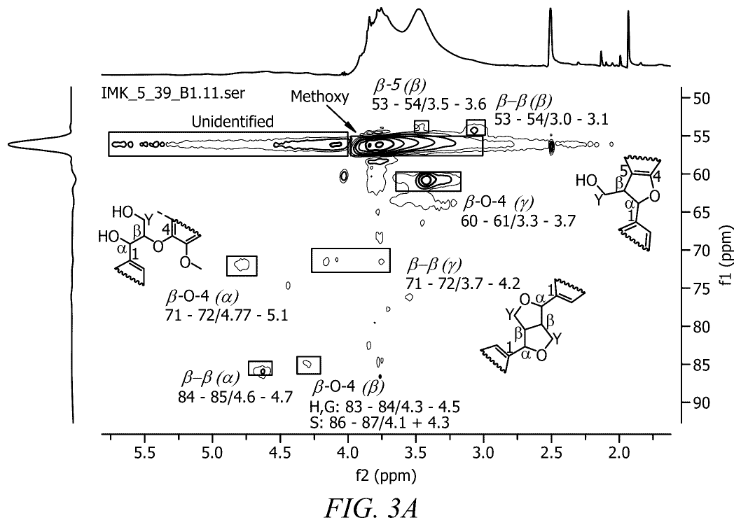

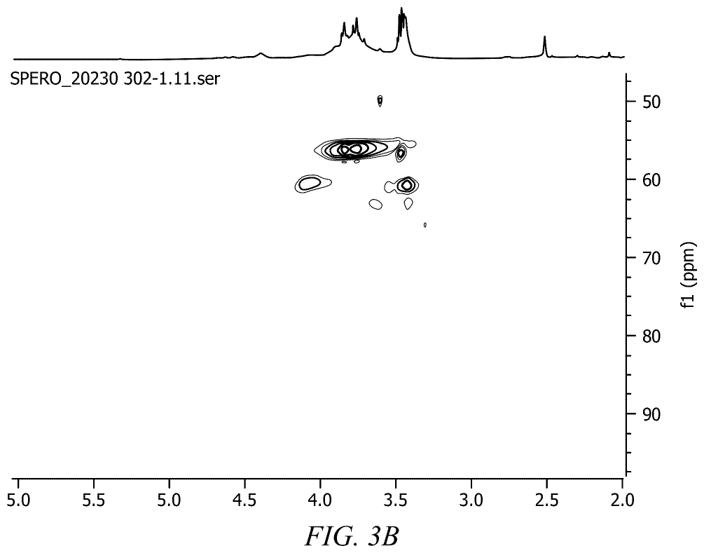

In some embodiments, the obtained bio-phenol products are directly used for epoxidation reactions without further purification. In some embodiments, the obtained bio-phenol products are analyzed by Gel Permeation Chromatography (GPC), 2D ¹H/¹³C HSQC Nuclear Magnetic Resonance Spectroscopy (NMR), and high-pressure liquid chromatography (HPLC). The GPC analysis shows the average Mₙ and the polydispersity index (PDI) of the bio-phenols. The 2D HSQC NMR analyzes the chemical structures of bio-phenols. In some embodiments, the NMR results indicates a full cleavage of lignin chemical linkages that results in small bio-phenol molecules which have Mₙ≤300 Da (FIG. 3), or Mₙ≤300 Da, or Mₙ≤500 Da, or Mₙ≤750 Da. In some embodiments, the obtained bio-phenols in this disclosure still contain some carbon-carbon (C—C) or carbon-oxygen-carbon (C—O—C) bond linkages which contribute to the higher Mₙ bio-phenols. HPLC analysis of the bio-phenols indicates lignin monomeric phenols are not the major product. In some embodiments, the solid residues (step (10) of this method) are washed by acetone and collected some soluble fractions. Analysis by GPC and 2D HSQC NMR of those acetone-soluble fractions indicates they are the heavy bio-phenols which have average Mₙ up to 60000 Da. Notably, this heavy bio-phenols extracted from solid residue can also be used for making epoxides. However, the resulting epoxides are very viscous. In some embodiments, the bio-phenols and solid residues are also used for elemental analysis. Compared to the intact kraft lignin (2.3% sulfur content or higher), the sulfur content is reduced by ≥50% in the bio-phenols. The procedures for GPC, HPLC, 2D HSQC NMR, and elemental analysis of bio-phenols, solid residues, and other lignin-derived products are well-known in published articles and herein are not discussed in this disclosure.

In some embodiments the spent catalysts including but not limited to Ni/C and Ni/Al₂O₃ can be regenerated by oxidative treatment at elevated temperatures, fully restoring the activity of the catalyst, allowing its reuse or recycling. This process relates to a method that reuses/regenerates the spent catalyst from the previous catalytic lignin depolymerization reaction. The catalytic reaction condition with recycled/regenerated catalyst stays the same as using a fresh catalyst which has been discussed in the above section.

Inductively coupled plasma optical emission spectroscopy (ICP-OES) is used to analyze fresh, spent, recycled, and regenerated Ni catalysts and indicates no Ni leaching from the surface of the catalyst during reaction with technical lignin. By which, no Ni leaching to the bio-phenols during reaction and thus no further metal contamination from catalyst to the resulting epoxy resin. The ICP-OES analysis is a well-known technology for trace metal analysis and thus the procedures for ICP-OES is not discussed in this disclosure.

This process relates to a method that converts bio-phenols over a TBAB catalyzed epoxidation reaction to produce bio-based epoxides. In some embodiments, intact kraft lignin is used for epoxidation. In some embodiments, the fractionated lignin by different solvents is used for epoxidation. In some embodiments, the bio-phenols obtained from different reaction conditions are used for epoxidation. In all cases of this disclosure, the epoxidation reaction conditions and steps remain the same or similar. In some embodiments, bio-based ECH are used to improve the bio-content of the epoxides and its corresponding epoxy resins.
(1) Load the lignin derived feedstock materials with 1-100× amount (by weight) of ECH and 1-75% (by weight) of TBAB into reaction vessel with continuous stirring at 1-2500 rpm.
(2) Heat the reaction mixture to 20-200° C. and held at the desired temperature for 0.1-6 h.
(3) After 0.1-6 h reaction, load 0.1-50% (by weight of ECH) of NaOH with small amount of water to the reaction mixture.
(4) After addition of aqueous (aq) NaOH, keep the reaction mixture at 20-200° C. for another 0.1-6 h.
(5) After the second 0.1-6 h, remove the reaction from heating but maintain the stirring between 1-2500 rpm.
(6) Once the reaction mixture is cooled to room temperature, 1-50 times (by volume of reaction mixture in ECH) of acetone is added to the reaction mixture.
(7) Filter the reaction mixture and collect the filtrate. The precipitate on filter contains waste NaCl.
(8) Remove the excess ECH and dry the acetone from filtrate by distillation or evaporation.
(9) The dried bio-based epoxides are collected as a blackish or dark brown colored material.

In some embodiments, the dried bio-based epoxides are free-flowing liquid which is due to its low viscosity (1-1500 cSt). In some embodiments, the dried bio-based epoxides are not a free-flowing liquid, instead, they show appearance as a gel or semi-solid materials. In all cases of the bio-based epoxides of this disclosure are able to be further upgraded to bio-based epoxy resins.

### Method of Bio-Based Epoxy Resin Synthesis

This process relates to a synthetic strategy for preparing bio-based epoxy resin from the described bio-based epoxides in the above section. In some embodiments, the bio-based epoxy resin is synthesized with 100% bio-based epoxides. In some embodiments, the bio-based epoxy resin is made of the blended mixture of bio-based epoxides and commercially available epoxies including but not limited to DGEBA at various ratios (weight ratio—wt/wt). In some embodiments, the bio-based epoxy resin is cured with an amine hardener including but not limited to Jeffamine-230, DETA, IPDA, or DDPS. In some embodiments, the bio-based epoxy resin is cured with IPDA. In some embodiments, the curing temperature remains low between 20-100° C. In some embodiments, the curing temperature is set to higher between 100-175° C. In some embodiments, a silicone mold is used to shape the bio-based epoxy resin. In some embodiments, metal molds, such as aluminum mold, or plastic mold, such as polytetrafluoroethylene (PTFE) mold are used to shape the bio-based epoxy resin.

In some embodiments, if epoxy resin synthesis uses a blended mixture between bio-based epoxides and DGEBA, the bio-based epoxides and DGEBA can be pre-mixed before mixing with curing agent. In some embodiments, if the viscous bio-based epoxides are used, to ensure the well mixing of bio-epoxides with either/both of DGEBA and curing agent, the viscous bio-based epoxides can be pre-heated at mild temperature between 20-100° C. to lower its viscosity before mixing with a curing agent. The synthesized bio-based epoxy resin is analyzed by dynamic mechanical analysis (DMA) for T_{g} and storage modulus measurements. The procedures of DMA analysis are well established and discussed in literature. In this disclosure, the analysis of DMA method is not discussed in detail.

### EXAMPLES

The disclosure having been generally described, the following examples are given as particular embodiments of the disclosure and to demonstrate the practice and advantages thereof. It is understood that the examples are given by way of illustration and are not intended to limit the specification or the claims in any manner.

### Example 1

### Lignin Fractionation by Organic Solvents

A sample of 50 g kraft lignin was loaded to a 1 L round bottom flask with 500 mL of EtOAc. The mixture was stirred at 500 rpm with magnetic stir bar. The round bottom flask was heated in a water bath at mild temperature around 40° C. After 2 h of heating, the mixture was poured through a filter paper in a Buchner funnel. The filtration was done under vacuum. Additional 100 mL EtOAc was used to wash the solid lignin cake on filter paper. The filtrate and additional EtOAc wash were combined into a 1 L round bottom flask. The round bottom flask was then attached to a rotavapor. The water bath of rotavapor was pre-set to 60° C. to warm the EtOAc solution in the round bottom flask. The vacuum of the rotavapor was set to −0.7 bar. The EtOAc solvent was fully removed from the round bottom flask which left the solid lignin fractions in the round bottom flask. The EtOAc-soluble lignin fraction was collected for further epoxidation reaction. The lignin fractionation by other organic solvents, such as EtOH and acetone, was performed similarly as Example 1 of this disclosure. The condition of rotavapor for drying EtOH and acetone was slightly tuned according to their boiling points.

### Example 2

### Production of Bio-Phenols From Kraft Lignin Over NI Catalyst

A sample of 5 g kraft lignin, 1 g Ni/C catalyst, and 40 mL 90% EtOH (EtOH/H₂O 9:1 v/v) were loaded to a 100 mL stainless-steel Parr batch reactor. The reactor was well sealed. The reaction was stirred by a mechanical stirring at 300 rpm. The reactor was then purged by N₂ gas three times and charged with 450 psi N₂. Prior to heating, the stirring rate was increased to 500 rpm. The heating process was programmed by an auto controller. The reactor was first heated to 200° C. and then held at 200° C. for 12 h. After 12 h reaction, the reactor was cooled to room temperature. The remaining gas pressure was first vented through a gas outlet. Then the reaction mixture was filtered through vacuum filtration. The solid residue was collected on filter paper while the dissolved bio-phenol products were collected in filtrate. An additional 100 mL EtOH was used to wash the solid residue on filter paper. The filtrate was then combined with the 100 mL EtOH wash in a 500 mL round bottom flask. The round bottom flask was then attached to a rotavapor. The water bath of rotavapor was pre-set to 80° C. to warm the product mixture. The vacuum pressure of rotavapor was first set to −0.6 bar to remove EtOH. Then the vacuum pressure was lowered to −0.8 bar to completely dry the remaining water. The dried bio-phenol products were collected in the round bottom flask. The yield of soluble bio-phenol products from the reaction was calculated by dividing its dry mass after solvent removal by the mass of the input kraft lignin. By which, under condition of Example 2, the yield of bio-phenols was up to 70wt % based on starting kraft lignin. The solid residue was dried together with Ni/C catalyst on filter paper. The solid mixture and filter paper was dried in an oven at 70° C. over night. The overall mass balance was calculated by the mass ratio between recovered total mass of bio-phenols, catalyst, and solid residue over the total input mass of kraft lignin and Ni/C catalyst. The mass balance for reaction described in Example 2 was determined at 91%. The missing mass could mainly attribute to the formation of gas phase products which were vented to fume hood after the reaction and thus was not counted into the mass balance. The production of bio-phenols with other reaction medium, such as 90% MeOH, 90% EtOH with 10% acetone, and 90% MeOH with 10% acetone were performed under similar reaction conditions with comparable reaction workup steps described in this Example 2. For example, of a 12 h catalytic reaction at 200° C. with the same loading of catalyst, the 90% MeOH with 10% acetone medium gave the highest yield of bio-phenol, which was up to 90%. The production of bio-phenol with Ni/Al₂O₃ was performed under similar reaction with comparable reaction workup steps described in this Example 2. The bio-phenols obtained in Example 2 at all reaction conditions were collected for further epoxidation reaction with ECH for produce bio-based epoxides. Notably, the sulfur content within bio-phenols was reduced to less than 50% of the original sulfur content in kraft lignin.

### Example 3

Production of Bio-Phenols From Continuous Flow Reactor A sample of 100 g kraft lignin and 800 mL 90% EtOH (EtOH/H₂O 9:1 v/v) were loaded to a 2 L stainless-steel tank. The stainless-steel tank was pre-heated to 40° C. and equipped with mechanical stirring at 600 rpm. The lignin-EtOH mixture was stirred at 40° C. for 30 min. During this process, 60% (wt/wt) of the input kraft lignin was pre-dissolved in the 90% EtOH. Then the solution was pumped to pass through a filter with 2.5 μm pore size to eliminate undissolved lignin particles from the homogeneous solution. Following the filtration, the lignin solution was continuously pumped through a fixed-bed stainless-steel flow reactor at 35 mL/min flow rate. The fixed-bed flow reactor was purged and pressurized to 450 psi under N₂. A portion of 100 g Ni/Al₂O₃ catalyst was packed in a 100 cm length and 10 cm diameter cylindrical catalyst bed and pre-heated to 200° C. prior to pumping the lignin feed. After a 4 h residence time, the pre-dissolved kraft lignin was fully converted to bio-phenols. The product stream was continuously pumped from catalyst bed through a heat exchanger and cooled to room temperature. The cooled product solution was collected at the outlet stream from the flow reactor in a stainless-steel tank. After solvent evaporation, a dry mass of 55 g bio-phenols was obtained. The yield of bio-phenols from pre-dissolved kraft lignin was determined at 92% (55 g out of 60 g dissolved lignin) while the overall yield of bio-phenols counted from initial input lignin was 55% (55 g out of 100 g kraft lignin). The overall mass balance was determined up to 95%. One missing mass was due to a few percentages of suspended solid formed during reaction and deposited to the catalyst bed. The other missing mass could attribute to the formation of undetermined gas phase products. The gaseous byproducts were depressurized and vented from the flow reactor. The production of bio-phenols from flow reactor were also investigated with other reaction medium, such as 90% MeOH, 90% EtOH with 10% acetone, and 90% MeOH with 10% acetone. The overall yield of bio-phenols could be improved to 90% in 90% MeOH with 10% acetone medium under the same reaction condition described in Example 3. This was due to an improved dissolution of kraft lignin in the reaction medium. By which, nearly 90% of kraft lignin was pre-dissolved into the 90% MeOH×10% acetone medium and pumped for catalysis through flow reactor. Regardless of the type of reactor (batch vs. flow) used for lignin catalysis in this disclosure, quality of the resulting bio-phenols appeared the same both chemically and physically.

### Example 4

### Extraction of Heavy Bio-Phenols

The heavy bio-phenols were produced during the catalytic lignin depolymerization reaction described in Example 2. The heavy bio-phenols gave higher average Mₙ and showed less solubility in the reaction medium. Thus, the heavy bio-phenols usually stayed with the solid residue following catalysis. In this disclosure, the heavy bio-phenols were found to be soluble in neat acetone. A portion of 5 g dry solid residues collect from the reaction products described in Example 2 with any kind of reaction medium was placed in a 250 ml beaker. A portion of 50 mL acetone was added to the solid residues. The mixture was stirred for 15 min at 300 rpm. After that, the mixture was filtered through vacuum filtration. The filtrate was collected and the solid on filter paper was transferred back to the 250 mL break and the 50 mL acetone wash was repeated. The acetone wash and vacuum filtration of the solid residue was repeated 3 times in total. All the filtrates were collected and combined into a 500 mL round bottom flask. The round bottom flask was then attached to a rotavapor. The temperature of water bath was pre-set to 50° C. to warm the acetone solution. The vacuum pressure of rotavapor was set to −0.5 bar to remove acetone. The fully dried heavy bio-phenols remained in the round bottom flask after the removal of acetone. The average percentage of heavy bio-phenol was determined to be around 20% of the total lignin solid residue described in Example 2. The remaining 80% of solid residue stayed insoluble in any kinds of common organic solvents, including but not limited to MeOH, EtOH, hexane, toluene, dichloromethane (DCM), chloroform, dimethyl sulfoxide, and dimethylformamide, etc. The remaining 80% insoluble solid residue was analyzed by carbon/hydrogen/nitrogen (C/H/N) elemental analysis which gave C_{65%}/H_{5%}/N_{0.4%}. The missing percentages of elements could attribute to oxygen, sulfur and other trace elements.

### Example 5

### Recycling of NI Catalysts

The spent catalyst was separated from the dried solid residues described in Example 2 or 3. A portion of 1.4 g dried spent Ni catalysts were collected and placed in a 100 ml beaker. The spent catalyst was first washed with 15 mL water and sonicated for 10 min. The water wash was carefully removed from catalyst. The water wash was repeated three times. After that, the spent catalyst was washed with 15 mL acetone and sonicated for 10 min. The acetone wash liquid was carefully removed from catalyst. The acetone was also repeated three times. At the last acetone wash, the catalyst-acetone mixture was separated by a vacuum filtration. The washed catalyst was collected on the filter paper while the acetone wash liquid was obtained in filtrate and discarded. The washed catalyst was transferred to a clean 50 ml beaker and dried in oven at 70° C. for 12 h. The dried recycled catalyst was weighed which was averaged about 1.1-1.2 g. Compared to the input catalyst in Example 2, there was still some mass gaining to the recycled catalyst. This was due to some coking or char formation to the catalyst surface that was hardly removed from catalyst surface. Notably, the mass gaining by coking and char formation could be accumulated to the catalyst surface if the catalyst was only recycled by the wash treatment described in Example 4. Overall, after the second recycle of the spent catalyst, the yield of bio-phenols could be decreased by 20-40%. In this disclosure, both Ni/C and Ni/Al₂O₃ were recycled by the same treatments described in Example 5.

### Example 6

### Catalyst Regeneration

The catalyst recycling by solvent wash described in Example 5 would not facilitate a long-term reuse of the spent catalyst. Thus, the catalyst regeneration method was studied. A portion of 1.4 g dried spent Ni/Al₂O₃ was collected from Example 2. The spent Ni/Al₂O₃ was first treated by the same water-acetone wash steps described in Example 5. After the washed Ni/Al₂O₃ was dried in an oven, the spent Ni/Al₂O₃ was then transferred into a 25 mL crucible and placed in a calcination furnace. The furnace was programmed to heat to 375° C. at a ramping rate of 2° C./min and held for 1-2 h under air. After that, the furnace was cooled to room temperature. The regenerated Ni/Al₂O₃ was collected in the crucible. There were some grey colored ashes in the crucible and on the surface of regenerated Ni/Al₂O₃ which could be easily removed by blowing air to the catalyst. The regenerated Ni/Al₂O₃ was weighed at around 0.95-1 g which indicated a complete recovery of input Ni/Al₂O₃ and a sufficient removal of coking or char from the catalyst surface. The regenerated Ni/Al₂O₃ described in Example 6 was ready for the catalytic depolymerization of kraft lignin to produce bio-phenols which gave the same yield and mass balance of products as the fresh Ni/Al₂O₃. The regenerated Ni/Al₂O₃ was also analyzed by ICP-OES which indicated no Ni leaching from the catalyst support under the recycling and regeneration conditions described in both Example 5 and 6.

### Example 7

### Production of Bio-Based Epoxides

A portion of 5 g bio-phenols generated from Example 2 or 3 was mixed with 50 mL ECH and 0.5 g TBAB in a 500 mL round bottom flask. The epoxidation mixture was stirred by magnetic stir bar at 350 rpm. The mixture was heated in an oil bath at 80° C. for 3 h. After that, 2.5 g NaOH and 2.5 mL deionized H₂O were added to the epoxidation mixture. The reaction mixture was kept at 80° C. for another 3 h. Then the reaction was cooled to room temperature. A portion of 200 mL acetone was charged to the reaction mixture with continuous stirring at 350 rpm for 15 min. During the stirring, NaCl was precipitated from organic reaction mixture. The mixture was filtered using vacuum filtration. The NaCl solid was collected on filter paper. The NaCl solid and filter paper were carefully washed by another 50 mL acetone. The filtrate and acetone wash were combined into a clean 500 mL round bottom flask. The round bottom flask was connected to a rotavapor. The water bath was first pre-set to 50° C. and the vacuum pressure was set to −0.6 bar to remove acetone. After that, the water bath was heated to 80° C. and vacuum was dropped to −0.8 bar to completely dry the ECH and water by evaporation. The dried liquid bio-based epoxides were collected as a product in the round bottom flask. The produced bio-based epoxides in this Example 7 were then used for epoxy resin synthesis. Similarly, the bio-phenols obtained from different reaction conditions described in Example 2 and 3, various lignin fractions described in Example 1, and heavy bio-phenols described in Example 4 were treated with the same epoxidation reaction conditions described in Example 7 to produce different bio-based epoxide molecules.

### Example 8

### Production of Epoxy Elastomer With 100% Bio-Based Epoxides

The bio-based epoxides that were obtained from Example 7 which was made with bio-phenols from 90% MeOH medium described in Example 2 was used. A 9 g portion of those bio-based epoxides were loaded to a round aluminum dish which had a diameter of 50 mm and thickness of 2 mm. A portion of 2 g Jeffamine-230 was added to the aluminum dish. The mixture was stirred for 20 min to ensure sufficient contact between epoxides and curing agent (Jeffamine-230). After that, the mixture was left in the fume hood at room temperature for 6 h. During the 6 h reaction, the resin was about 20% cured. Then the mixture was transferred to a 70° C. oven and heated for another 6 h. During the second 6 h reaction, the resin was about 60% cured. However, there was still obvious liquid in the mixture. Finally, the mixture was transferred into a 120° C. oven and heated for 2 h. The elastic epoxy resin was fully cured at 120° C. The bio-based epoxy elastomer was shaped by the round aluminum dish and could be removed from the dish easily when it was cooled to room temperature. The obtained elastomer gave bio-content up to 72%. The color of bio-based elastomer was dark brown with a smooth and shiny surface.

### Example 9

### Production of Epoxy Resin With Improved Hardness

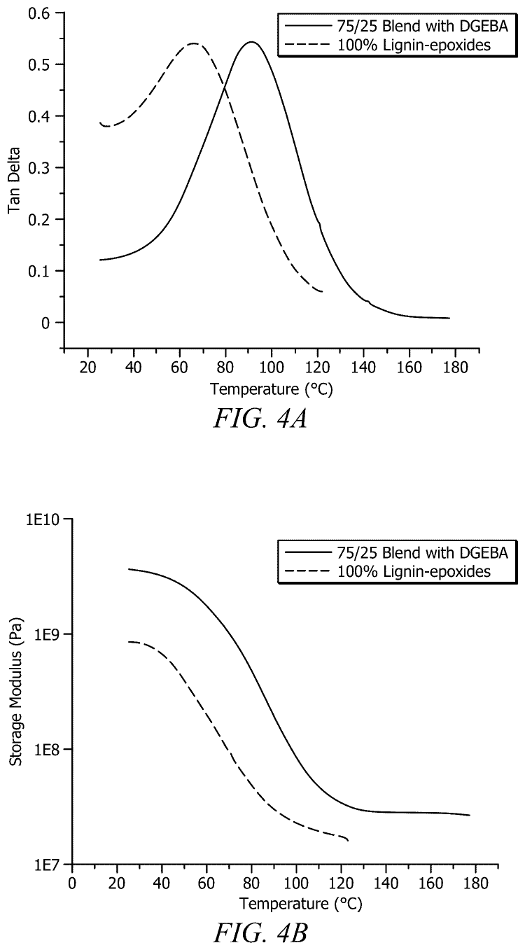

There were two ways to reduce the elasticity and increase the hardness of bio-based epoxy resin: (1) blend the bio-based epoxides with DGEBA, and (2) use different curing agents such as IPDA and DETA. In some cases of Example 9-(1), a portion of 4 g bio-based epoxides were mixed with an equal amount of DGEBA in a silicone mode. The mixture of epoxides was first stirred at room temperature for 10 min to ensure a complete mixing. Then a portion of 2 g Jeffamine-230 was added to the epoxides mixture and stirred for another 10 min to ensure the complete mixing of epoxides and curing agent. The mixture was placed in a fume hood at room temperature for 6 h and then transferred the mixture to a 70° C. oven for another 6 h. After that, the mixture was transferred to 120° C. oven for another 2 h. The cured epoxy resin could be carefully removed from silicone mold when it was cooled to 45° C. The obtained bio-based epoxy resin contained 37% bio-content. For other cases of Example 9-(1), a portion of 7.5 g bio-based epoxides were pre-mixed with 2.5 g DGEBA. Then a 1.7 g Jeffamine-230 was mixed with the epoxides and stirred for 10 min. The curing steps stayed the same as described above. The resulting bio-based epoxy resin gave up to 56% bio-content. In some cases of Example 9-(2), a portion of 10 g bio-based epoxides were well mixed with either 1.7 g IPDA or 1 g DETA in a silicone mold. The mixture was left in a fume hood at room temperature for 6 h. In contrast to the Jeffamine-230, the IPDA and DETA are more reactive. Thus, the resin cured by IPDA and DETA gave higher degree of curing at room temperature. After 6 h curing, the mixture was then transferred to a 70° C. oven to complete the curing process within another 6 h. For other cases of Example 9-(2), a portion of 7.5 g bio-based epoxides was first pre-mixed with 2.5 g DGEBA. After that, the epoxides were well mixed with either 1.3 g IPDA or 1.1 g DETA in a silicone mold. The mixture was first left in fume hood for 6 h and transferred to 70° C. for another 6 to complete the curing process. Notably, the bio-based epoxy resins obtained in Example 8 gave improved hardness which showed a range of T_{g} between 30-90° C. with wide range of storage modulus (FIG. 4). In addition to Example 9, if the bio-based epoxides were derived from lignin fractions in Example 1 or heavy bio-phenols in Example 4, the bio-based epoxides can be pre-heated at mild temperature around 30-40° C. to lower the viscosity before the resin synthesis. This was to ensure a good blending with DGEBA or sufficient mixing with curing agents.

### Example 10

### Formulation of Bio-Based Epoxy Resin for Specific Mechanical Properties

In order to meet specific targets of mechanical properties, such as Tg, storage modulus, and flame retardancy, several examples of bio-based epoxy resin formulation are presented in Example 10 and 11. As described in Example 9, the bio-based epoxides can be blended with DGEBA in its formulation and used with multiple curing agents. In particular, to produce an epoxy resin with Tg of 70° C. of higher and having minimum storage modulus of 1000 MPa (25° C.), the bio-based epoxides (100% bio-content) was cured solely with IPDA in a mass ratio of 1:0.17 (epoxides: IPDA wt/wt) under the same curing condition described in Example 9. To increase the Tg up to 90° C. or higher, a portion of DGEBA was pre-mixed with the bio-based epoxides in a mass ratio of 1:3 (DGEBA: bio-based epoxides, wt/wt). The resulting epoxides mixture gave 75% bio-content. Then the mixture of epoxides could follow the same curing procedure described in Example 9 with using the same ratio of IPDA (1: 0.17) for resin synthesis. The Tg of epoxy resin was investigated for further improvement to reach 115° C. or higher by formulating the bio-epoxides with 1:1 mass ratio with DGEBA, resulting 50% bio-content within the epoxides mixture, and cured with IPDA under the same condition mentioned in Example 9.

### Example 11

### Formulation Of Flame-Retardant Bio-Based Epoxy Resin

Bio-based epoxides were formulated to produce a flame-retardant epoxy resin in Example 11. The resin was synthesized by bio-based epoxides (100% bio-content) and cured with DDPS. In a silicon mold, bio-based epoxides were mixed with DDPS at a mass ratio of 1:0.25 (epoxides: DDPS wt/wt). The mixture was heated to 40° C. with 300 rpm stirring to ensure a comprehensive mixing between the two reactants. After a 15 min stirring at 40° C., the homogeneous gel-like mixture was transferred to a fume hood and left at room temperature for 6 h. During this period, the epoxides were partially cured with DDPS while air and gaseous byproducts were released from the mixture. Then the mixture was placed in a 70° C. oven to reach 80-90% degree of curing for another 6 h. Finally, the mixture was transferred to a 120° C. oven for 2 h to complete the curing process. The flame-retardant bio-based epoxy resin was obtained after the material was cooled to room temperature. Performance of flame retardancy of the resulting resin was analyzed by (1) American Society for Testing and Materials (ASTM) D3801-10 method for vertical burning test; (2) ASTM D2863-97 method for its limiting oxygen index (LOI); (3) international standards organization (ISO) 5660 method by cone calorimetry for combustion behavior; and (4) ISO 5659-2 method for its smoke density performance. Compared to a resin synthesized by petroleum-based DGEBA under the same condition as the bio-based resin with DDPS, the bio-based resin made in Example 11 showed high carbonization and flame retardant due to the results of an average 100% less of fire growth rate, 30% more of LOI, 25% lower of heat release rate, 50% less of smoke density, and 100% more of char residue. In some cases of the flame-retardant bio-based resin described in Example 11 were synthesized by 75% or 50% bio-content epoxides.

Having described various compounds, formulations, processes, and systems, various aspects can include, but are not limited to:
In a first aspect, a method comprises: contacting lignin with a catalyst to catalytically depolymerize lignin into low molecular weight fragments (Mₙ≤350 Da) that contain unbound phenolic hydroxyl groups.
A second aspect can include the method of the first aspect, wherein the lignin comprises technical lignin, kraft lignin, LignoBoost lignin, organosolv lignin, acid or base extracted lignin, steam explosion extracted lignin, hot water extracted lignin, or derivatives thereof.
A third aspect can include the method of the first or second aspect, wherein the depolymerized lignin products have hydroxyl number of 2 -12 mmol/g_{bio-phenol} or about 6-8 mmol/g_{bio-phenol}.
A fourth aspect can include the method of the first or second aspect, wherein the depolymerized lignin products have an average Mₙ of less than or equal to 350 Da or more than or equal to 700 Da or more than or equal to 1000 Da in different solvents, such as 90% EtOH, 90% EtOH with acetone, 90% MeOH, and 90% MeOH with acetone.
A fifth aspect can include the method of any one of the first to fourth aspects, further comprising: regenerating the catalyst by thermal oxidative treatment.
A sixth aspect can include the method of any one of the first to fourth aspects, further comprising: converting the depolymerized lignin without further purification to lignin-based organic epoxides by reaction with petroleum or bio-based or bio-attributed epichlorohydrin.
A seventh aspect can include the method of the sixth aspect, wherein the lignin-based organic epoxides are a free-flowing liquid at room temperature with a viscosity of 1-700 cSt or lower at temperature above 40° C. and can be blended with DGEBA or other commercial epoxides.
An eighth aspect can include the method of any one of the first to seventh aspects, wherein the depolymerized lignin products have an average Mn of more than or equal to 1000 Da, and wherein upon treatment of the depolymerized lignin products with epichlorohydrin yields lignin-based epoxides that have a viscosity in the range 700-1500 cSt.
A ninth aspect can include the method of the seventh or eighth aspect, wherein lignin-based epoxies are miscible with DGEBA (5-75 wt %) to give a free-flowing liquid at room temperature.
A tenth aspect can include the method of any one of the first to ninth aspects, wherein the lignin and depolymerized lignin products contain sulfur.
An eleventh aspect can include the method of any one of the first to tenth aspects, further comprising: obtaining a solid residue from the contacting; and obtaining process heavy bio-phenols with a Mₙ greater than 30000 Da by solvent washing of the solid residue.
In a twelfth aspect, a method comprises: contacting technical lignin with one or more organic solvents; fractioning the technical lignin based on the contacting to form lignin fractions; and epoxidizing one or more of the lignin fractions for resin synthesis to form lignin epoxide.
A thirteenth aspect can include the method of the twelfth aspect, where the lignin-derived epoxides contain sulfur.
In a fourteenth aspect, a method comprises: contacting kraft lignin with a catalyst; catalytically depolymerizing the kraft lignin with the catalyst; and reducing a sulfur content of the kraft lignin based on the catalytic depolymerization reaction.
In a fifteenth aspect, a method comprises: contacting lignin with a catalyst to depolymerize at least a portion of the lignin; exhausting at least a portion of the catalyst in response to contacting the lignin with the catalyst; sequential washing the exhausting catalyst with water and organic solvents to rejuvenate the catalyst; and reusing the rejuvenated catalyst to depolymerize an additional portion of lignin.
A sixteenth aspect can include the method of the fifteenth aspect, wherein heavy bio-phenols can be epoxidized with epichlorohydrin to form high viscosity bio-based epoxides having a viscosity of greater than 700 cSt at 25° C.
In a seventeenth aspect, a method comprises: producing bio-based elastomers from lignin-derived epoxides cured with amine hardeners, such as Jeffamine-230, IPDA, DETA, and DDPS at room temperature to 120° C.
In an eighteenth aspect, a method comprises: producing bio-based rigid epoxy resin with wide range of mechanical properties, such as T_{g}<20° C. or 60-90° C., or 90-150° C., or >150° C. and storage modulus between 0.3-3 GPa.
In a nineteenth aspect, a method comprises: producing a formulation of bio-based epoxy resin with greater than 50 wt % bio-based content.
A twentieth aspect can include the method of any one of the first to nineteenth aspects, wherein the epoxy resin is BPA-free.
A twenty first aspect can include the method of any one of the first to twentieth aspects, wherein the bio-based epoxy resin is produced using 100% bio-based epoxides.
In a twenty second aspect, a reactor system comprises: a lignin dissolution vessel configured to contact lignin with a solvent; a catalytic reactor in fluid communication with the lignin dissolution vessel, wherein the catalyst reactor comprises a catalyst bed configured to contact soluble lignin from the lignin dissolution vessel at elevated temperature and pressure; and a separator configured to separate product bio-phenols from the solvent are recycle the solvent to the lignin dissolution vessel.
A twenty third aspect can include the method of any one of the first to twenty first aspects, wherein the cured bio-based epoxy resin, such as cured by DDPS, is flame retardant.
In a twenty fourth aspect, a method comprises: producing a formulation of flame-retardant bio-based epoxy resin with limiting oxygen index >29%, total heat release rate <70 megajoule per square meters (MJ/m²), time to ignition >4 seconds, total smoke production <25 m².

It is to be further understood that the present description is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present systems and methods. It must be noted that as used herein and in the appended claims (in this application, or any derived applications thereof), the singular forms “a,” “an,” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this description belongs. Preferred methods, techniques, devices, and materials are described, although any methods, techniques, devices, or materials similar or equivalent to those described herein may be used in the practice or testing of the present systems and methods. Structures described herein are to be understood also to refer to functional equivalents of such structures. The present systems and methods will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims may be formulated in this Application or of any further Application derived therefrom, to particular combinations of features, it should be understood that the scope of the disclosure also includes any novel feature or any novel combination of features disclosed herein either explicitly or implicitly or any generalization thereof, whether or not it relates to the same systems or methods as presently claimed in any Claim and whether or not it mitigates any or all of the same technical problems as do the present systems and methods.

Features which are described in the context of separate embodiments may also be provided in combination in a single embodiment. Conversely, various features which are, for brevity, described in the context of a single embodiment, may also be provided separately or in any suitable sub-combination. The Applicants hereby give notice that new claims may be formulated to such features and/or combinations of such features during the prosecution of the present Application or of any further Application derived therefrom.

1. A method comprising:
contacting lignin with a catalyst; and
based on the contacting, catalytically depolymerizing at least a portion of the lignin into low molecular weight fragments, wherein the low molecular weight fragments have Mₙ≤350 Da, and wherein the low molecular weight fragments comprise unbound phenolic hydroxyl groups.

2. The method of claim 1, wherein the lignin comprises technical lignin, kraft lignin, LignoBoost lignin, organosolv lignin, acid or base extracted lignin, steam explosion extracted lignin, hot water extracted lignin, or derivatives thereof.

3. The method of claim 1, wherein the low molecular weight fragments have a hydroxyl number of 2 -12 mmol/g_{bio-phenol} and an average Mₙ of less than or equal to 350 Da.

4.-5. (canceled)

6. The method of claim 1, further comprising:
converting the low molecular weight fragments without further purification to lignin-based organic epoxides by reaction with petroleum, bio-based, or bio-attributed epichlorohydrin.

7. The method of claim 6, wherein the lignin-based organic epoxides are a free-flowing liquid with a viscosity of less than 700 cSt at temperature at or above 40° C., where the lignin-based organic epoxides can be blended with DGEBA.

8. (canceled)

9. The method of claim 1, wherein the low molecular weight fragments have an average Mₙ of more than or equal to 1000 Da, and wherein the low molecular weight fragments, upon treatment of with epichlorohydrin, yield lignin-based epoxides having a viscosity in a range of 700-1500 cSt.

10. The method of claim 7, wherein lignin-based epoxies are miscible with DGEBA in an amount of between 5 -75 wt % at room temperature.

11. (canceled)

12. The method of claim 1, further comprising:
obtaining a solid residue from the contacting;
washing the solid residue with the solvent; and
obtaining process heavy bio-phenols with a Mₙ greater than 30000 Da by washing the solid residue with the solvent.

13. The method of claim 1, wherein the lignin is kraft lignin, wherein the kraft lignin comprises sulfur, and wherein the method further comprises:
reducing a sulfur content of the kraft lignin based on the catalytic depolymerization and wherein the lignin and the low molecular weight fragments contain sulfur.

14. The method of claim 1, further comprising:
exhausting at least a portion of the catalyst in response to contacting the lignin with the catalyst to form an exhausted catalyst;
sequential washing the exhausted catalyst with water and organic solvents to regenerate the catalyst or regenerating the catalyst by thermal oxidative treatment to form a regenerated catalyst;
and
reusing the regenerated catalyst to depolymerize an additional portion of lignin.

15. The method of claim 1, further comprising:
obtaining heavy bio-phenols with Mₙ of 35,000-40,000 Da or up to 60,000 Da from the contacting; and
epoxidizing the heavy bio-phenols with epichlorohydrin to form high viscosity liquid or solid bio-based epoxides having a viscosity of greater than 700 cSt at 25°C.

16. A method comprising:
contacting technical lignin with one or more organic solvents; and
fractioning the technical lignin based on the contacting to form lignin fractions; and
epoxidizing one or more of the lignin fractions for resin synthesis to form lignin epoxide.

17. The method of claim 16, where the lignin epoxide contains sulfur.

18. The method of claim 6 wherein
the lignin-derived epoxides are cured with one or more amine hardeners, wherein the one or more amine hardeners comprise Jeffamine-230, IPDA, DETA, and DDPS, and wherein the curing occurs at a temperature between about 25 C to about 120 C; and
producing bio-based elastomers from the lignin-derived epoxides cured with the amine hardeners.

19. The method of claim 18 comprising:
producing a bio-based epoxy resin, wherein the epoxy resin has a T_{g} between about 20° C. to 150° C., and a storage modulus between 0.3-3 GPa.

20. The method of claim 19, further comprising:
producing the bio-based epoxy resin with greater than 50 wt % bio-based content.

21. The method of claim 19, wherein the epoxy resin is BPA-free and wherein the epoxy resin is produced using 100% bio-based epoxides.

22. (canceled)

23. The method of claim 19, further comprising:
curing the epoxy resin to form a cured resin, wherein the cured resin is flame retardant.

24. The method of claim 19, further comprising:
producing, using the epoxy resin, a formulation of flame-retardant bio-based epoxy resin, wherein the flame-retardant bio-based epoxy resin has a limiting oxygen index >29%, a total heat release rate <70 MJ/m², a time to ignition >4 seconds, and a total smoke production <25 m².

25. The method of claim 1, wherein the catalytic depolymerization of lignin occurs in a reactor system comprising:
a lignin dissolution vessel configured to contact lignin with a solvent;
a catalytic reactor in fluid communication with the lignin dissolution vessel, wherein the catalyst reactor comprises a catalyst bed configured to contact soluble lignin from the lignin dissolution vessel at elevated temperature and pressure; and
a separator configured to separate product bio-phenols from the solvent are recycle the solvent to the lignin dissolution vessel.
